(19) United States
(12) Patent Application Publication
(10) Pub. No.: US 2025/0012728 A1
(43) Pub. Date: Jan. 9, 2025

(54) SURFACE-ENHANCED RAMAN SCATTERING (SERS)-BASED TEST KIT AND DETECTION METHOD FOR STAPHYLOCOCCUS AUREUS

(71) Applicants: JIMEI UNIVERSITY, Xiamen (CN); JIANGSU UNIVERSITY, Zhenjiang (CN)

(72) Inventors: Quansheng CHEN, Xiamen (CN); Afang ZHU, Zhenjiang (CN); Yi XU, Xiamen (CN); Tianhui JIAO, Xiamen (CN); Wenya WEI, Zhenjiang (CN); Zhen WANG, Zhenjiang (CN); Songguang ZHAO, Zhenjiang (CN)

(21) Appl. No.: 18/897,054

(22) Filed: Sep. 26, 2024

(30) Foreign Application Priority Data
Jul. 19, 2024 (CN) 202410970199.4

(51) Int. Cl. G01N 21/65; C12Q 1/682; C12Q 1/6834; C12Q 1/689

(52) U.S. Cl. CPC G01N 21/658; C12Q 1/682; C12Q 1/6834; C12Q 1/689

(57) ABSTRACT

A surface-enhanced Raman scattering (SERS)-based test kit for Staphylococcus aureus includes a magnetic nanosphere, gold/silver core-shell nanoparticles (Au—Ag NPs), Au nanoparticles (Au NPs), a SERS signaling molecule, a rolling circle amplification (RCA) template, a target aptamer, a complementary deoxyribonucleic acid (cDNA) and a single-stranded deoxyribonucleic acid (ssDNA). A detection method for S. aureus using the SERS-based test kit is also provided.

### REFERENCE TO AN ELECTRONIC SEQUENCE LISTING

The contents of the electronic sequence listing (Name: SequenceListing.xml; Size: 4,550 bytes; and Date of Creation: Aug. 8, 2024) is herein incorporated by reference in its entirety.

### CROSS-REFERENCE TO RELATED APPLICATIONS

This application claims the benefit of priority from Chinese Patent Application No. 202410970199.4, filed on Jul. 19, 2024. The content of the aforementioned application, including any intervening amendments made thereto, is incorporated herein by reference in its entirety.

### TECHNICAL FIELD

This application relates to bio-detection technology, and more particularly to a surface-enhanced Raman scattering (SERS)-based test kit and detection method for Staphylococcus aureus.

### BACKGROUND

Biosensing technology has been widely applied in biomedicine, environmental monitoring, and food safety monitoring. However, the differences in the structure and properties of biomolecules themselves make the detection difficult, especially for those biomolecules with low concentrations, and traditional methods often fail to achieve the sensitive and specific detection.

Surface-enhanced Raman scattering (SERS) has emerged as an advanced spectroscopic analysis technique, which can achieve the label-free, highly-sensitive and nondestructive detection of biomolecules at the molecular level based on the significant enhancement effect of metal nanostructures on Raman scattering signals. However, in the practical applications, the SERS technique suffers from unstable signal strength and high interference from background noise. Therefore, it is necessary to further improve the detection sensitivity and specificity of the SERS technology to achieve the highly efficient, sensitive and specific detection of biomolecules.

### SUMMARY

In view of this, an object of the present disclosure is to provide a surface-enhanced Raman scattering (SERS)-based test kit and detection method for Staphylococcus aureus, which have significantly improved detection sensitivity and accuracy.

In a first aspect, this application provides a SERS-based test kit for Staphylococcus aureus, comprising:
a magnetic microsphere;
a gold-silver core-shell nanoparticle (Au—Ag NP);
a gold nanoparticle (Au NP);
a SERS signaling molecule;
a rolling circle amplification (RCA) template;
a target aptamer;
complementarity deoxyribonucleic acid (cDNA); and
a single-stranded deoxyribonucleic acid (ssDNA);
wherein a nucleotide sequence of the RCA template consists of SEQ ID NO: 1 with 5′ end modified with a phosphate group and 3′ end modified with a hydroxyl group;
a nucleotide sequence of the target aptamer consists of SEQ ID NO: 2;
a nucleotide sequence of the cDNA consists of SEQ ID NO: 3 with 5′ end modified with a sulfhydryl group; and
a nucleotide sequence of the ssDNA consists of SEQ ID NO: 4 with 5′ end modified with an amino group.

In some embodiments, the magnetic nanosphere is an iron nanosphere.

In some embodiments, a particle size of the Au—Ag NP is 15-25 nm.

In some embodiments, a particle size of the magnetic nanosphere is 165-175 nm.

In some embodiments, a particle size of the Au NP is 10-20 nm.

In some embodiments, the SERS signaling molecule is selected from the group consisting of 4-nitrobenzenethiol, 4-mercaptobenzoic acid, 4-aminobenzenethiol and a combination thereof.

In some embodiments, the SERS-based test kit further comprises at least one of T4 DNA ligase, deoxyribonucleotide triphosphates (dNTPs) and Phi29 DNA polymerase.

In a second aspect, this application provides a SERS-based detection method for Staphylococcus aureus using the SERS-based test kit, comprising:
(1) charging the magnetic nanosphere positively to obtain a positively-charged magnetic nanosphere;
(2) dispersing the positively-charged magnetic nanosphere in ultrapure water to obtain a first dispersion, and adding the Au—Ag NP to the first dispersion followed by ultrasonic reaction for 2.5-3.5 h and magnetic separation to obtain an Au—Ag nanoparticle-modified nanosphere;
(3) ultrasonically dispersing the Au—Ag nanoparticle-modified nanosphere in a first phosphate buffered saline to obtain a second dispersion; adding the cDNA to the second dispersion followed by incubation at 25-40° C. for 6-10 h and magnetic separation to collect a separated product; ultrasonically dispersing the separated product into a second phosphate buffered saline to obtain a third dispersion; and adding the target aptamer to the third dispersion followed by incubation at 25-40° C. for 30-60 min and magnetic separation to obtain a bio-functionalized Au—Ag nanoparticle-modified nanosphere;
(4) reacting the Au NP with the SERS signaling molecule under stirring for 20-40 min followed by centrifugal separation to obtain a signaling molecule-modified Au NP (Au-SM);
(5) reacting the Au-SM with a 3-aminopropyltriethoxysilane aqueous solution under stirring for 10-30 min to obtain a reaction product, and reacting the reaction product with a sodium silicate solution under stirring at 85-95° C. for 1.5-2.5 h followed by standing away from light for 10-12 h, and centrifugal separation to obtain a silica-coated Au-SM (Au-SM/SiO₂);
(6) ultrasonically mixing the Au-SM/SiO₂ with a 3-aminopropyltriethoxysilane aqueous solution for 30-40 min to obtain an amino-modified Au-SM/SiO₂, and reacting the amino-modified Au-SM/SiO₂ with a cyanuric chloride solution at room temperature under stirring for 2-3 h followed by addition of the ssDNA and incubation at 25-40° C. for 6-10 h to obtain a ssDNA-linked Au-SM/SiO₂; and
(7) incubating the bio-functionalized Au—Ag nanoparticle-modified nanosphere obtained in step (3) with a to-be-detected sample containing Staphylococcus aureus at 20-40° C. for 0.5-2 h followed by magnetic separation to obtain a first separated product; adding the RCA template, a T4 DNA ligase and a T4 DNA ligase reaction buffer to the first separated product to obtain a reaction system; adjusting the reaction system to pH 7-8 followed by reaction at 20-40° C. for 1-3 h and magnetic separation to obtain a second separated product; adding dNTPs, a Phi29 DNA polymerase and a Phi29 DNA polymerase reaction buffer to obtain an incubation system; incubating the incubation system at 20-40° C. for 0.5-2 h followed by addition of the ssDNA-linked Au-SM/SiO₂, incubation at 20-40° C. for 0.5-2 h and magnetic separation to obtain a third separated product; and detecting the third separated product using a Raman spectrometer.

In some embodiments, in step (7), the reaction system is adjusted to pH 7.3-7.5.

In some embodiments, in step (7), the reaction system is reacted at 35-40° C.

In some embodiments, in step (7), a final concentration of the Phi29 DNA polymerase in the incubation system is 0.4-0.7 U/μL.

In some embodiments, in step (7), a final concentration of the T4 DNA ligase in the reaction system is 4-7 U/μL.

In a third aspect, this application provides a biosensor, wherein raw materials for the preparation of the biosensor comprise the SERS-based test kit.

Compared to the prior art, this application has the following beneficial effects.

The SERS-based test kit of the present disclosure includes a magnetic nanosphere, an Au—Ag NP, an Au NP, SERS signaling molecules, an RCA template, a target aptamer, a cDNA, and an ssDNA. The magnetic nanosphere, the Au—Ag NP, the cDNA and the target aptamer can be combined to form a bio-functionalized Au—Ag nanoparticle-modified nanosphere, and the Au NP, the SERS signaling molecule and the ssDNA can be combined to form a SERS probe. Upon exposed to a sample containing S. aureus, the target aptamer in the bio-functionalized Au—Ag nanoparticle-modified nanosphere will be tightly bound to the S. aureus to form a stable complex, and the cDNA is released. Then the RCA template binds to the cDNA on the bio-functionalized Au—Ag nanoparticle-modified nanosphere to trigger RCA, so as to generate a large number of long single-stranded DNA repeat sequences with a specific repeat, thereby significantly increasing the copy number of the target DNA and providing abundant recognition sites for subsequent SERS detection. Subsequently, the SERS probe undergoes rapid and efficient molecular hybridization with the long single-stranded DNA repeat sequences based on complementary base pairing to further enhance the SERS signal.

In terms of the detection of S. aureus, the present disclosure optimizes the combination of the RCA template, the target aptamer, cDNA and ssDNA, where the target aptamer has high affinity and specificity to S. aureus, and only S. aureus can be stably bound to the target aptamer. The cDNA is released to initiate the RCA, allowing for a high detection specificity. The amplification product obtained using the RCA template has a high complementarity and affinity to the ssDNA, thereby improving the hybridization efficiency and detection sensitivity.

The RCA technology and the SERS-based detection technique have been successfully combined herein by optimizing the experimental conditions of the RCA and improving the design of the nanomaterials, which achieves the dual signal amplification, thereby improving the detection sensitivity, specificity and accuracy. This application has high sensitivity and specificity, and a low detection limit for the detection of S. aureus.

### BRIEF DESCRIPTION OF THE DRAWINGS

FIGS. 1A-1B are transmission electron microscope (TEM) images of gold/silver core-shell nanoparticles (Au—Ag NPs), where 1A: scale bar: 20 nm; and 1B: scale bar: 10 nm.

FIG. 2A schematically shows ultraviolet (UV) spectral properties of Au nanoparticles (Au NPs), Ag nanoparticles (Ag NPs), Au—Ag NPs and a mixture of Au NPs and Ag NPs.

FIG. 2B schematically shows the particle size distribution of Au—Ag NPs.

FIG. 3A is a TEM image of the prepared magnetic nanospheres.

FIG. 3B is a TEM image of the Au—Ag nanoparticle-modified nanospheres.

FIG. 4 schematically shows finite-difference time-domain data of an electric field strength distribution of the Au—Ag nanoparticle-modified nanospheres and Au NPs.

FIG. 5A illustrates comparison between the magnetic nanospheres and the Au—Ag nanoparticle-modified nanospheres in terms of the magnetic hysteresis loop.

FIG. 5B shows UV adsorption of magnetic nanospheres, Au—Ag nanoparticle-modified nanospheres and bio-functionalized Au—Ag nanoparticle-modified nanospheres.

FIG. 6A schematically shows TEM characterization data of Au NPs.

FIG. 6B schematically shows particle size distribution of the Au NPs.

FIG. 6C schematically shows TEM characterization data of Au-SM/SiO₂.

FIG. 6D schematically shows particle size distribution of the Au-SM/SiO₂.

FIG. 7 schematically shows a synthetic route of ssDNA-linked Au-SM/SiO₂.

FIG. 8A shows a typical SERS spectrum recorded by the signal amplification platform of the present disclosure during the detection of various concentrations of Staphylococcus aureus.

FIG. 8B illustrates a functional relationship between S. aureus concentration and SERS intensity.

FIG. 9A schematically shows the principle of rolling circle amplification (RCA).

FIG. 9B schematically shows the detection principle of a test kit of the present disclosure.

FIGS. 10A-10E are TEM images of Au—Ag nanoparticle-modified nanospheres obtained under different volumes of Au—Ag NPs, where A: 0 mL; B: 10 mL; C: 20 mL; D: 30 mL; and E: 40 mL.

FIG. 11A shows SERS spectra of Au—Ag nanoparticle-modified nanospheres obtained through addition of different volumes of Au—Ag NPs.

FIG. 11B schematically shows SERS intensity of Au—Ag nanoparticle-modified nanospheres obtained through addition of different volumes of Au—Ag NPs.

FIG. 12A schematically shows change of the SERS intensity versus pH of the RCA system.

FIG. 12B schematically shows change of the SERS intensity versus reaction temperature of the RCA.

FIG. 13A schematically shows optimization results of the amount of phi29 DNA polymerase.

FIG. 13B schematically shows optimization results of the amount of T4 DNA ligase.

FIG. 14A schematically shows optimization results of the RCA time.

FIG. 14B schematically shows optimization results of the hybridization time between the RCA product and the signal probe.

FIGS. 15A-15B schematically show test results of the specificity of the SERS-based detection method of the present disclosure for S. aureus.

### DETAILED DESCRIPTION OF EMBODIMENTS

Unless otherwise specified, the experimental methods in the following embodiments of the present disclosure for which specific conditions are generally performed in accordance with conventional conditions (Sambrook, et al. Molecular Cloning: A Laboratory Manual[M]. New York: Cold Spring Harbor Laboratory Press, 1989), or in accordance with the conditions recommended by the manufacturer. Various common chemical reagents used in the embodiments are commercially available.

Unless otherwise defined, all technical and scientific terms used in the present disclosure have the same meaning as commonly understood by those of ordinary skill in the art. The terms used in the description of the present disclosure are merely descriptive, and are not intended to limit the disclosure.

Terms “comprise” and “have”, and any variations thereof are intended to cover non-exclusive inclusion. For example, a process, method, apparatus, product or device containing a series of steps or elements is not limited to the listed steps or elements, but optionally also includes steps or element that are not listed, or optionally includes other steps or element that are inherent to the process, method, product or device.

As used herein, the phrase “a plurality of” means two or more. The “and/or” describes a relationship of the associated objects, and indicates the existence of three solutions, for example, “A and/or B” includes “A”, “B” and a combination thereof.

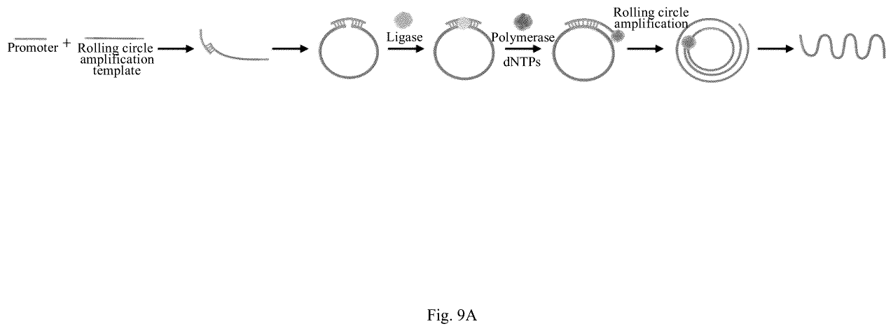

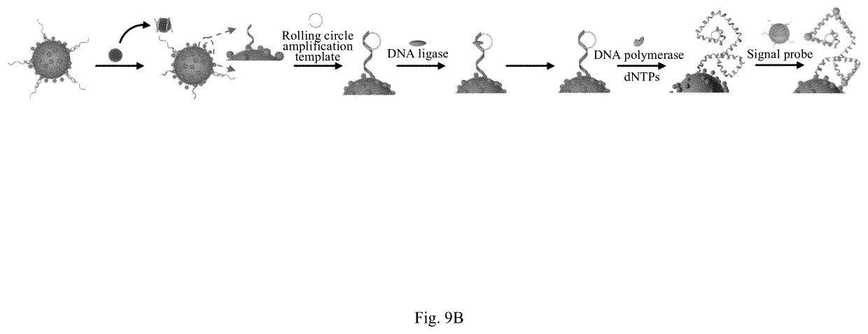

The principle of rolling circle amplification (RCA) is schematically shown in FIG. 9A, and the detection principle of the present disclosure is schematically shown in FIG. 9B. In the present disclosure, magnetic nanospheres, gold/silver core-shell nanoparticles (Au—Ag NPs), complementary deoxyribonucleic acid (cDNA) and target aptamer can be combined together to form bio-functionalized Au—Ag nanoparticle-modified nanospheres; and Au nanoparticles (Au NPs), surface-enhanced Raman scattering (SERS) signaling molecules and single-stranded deoxyribonucleic acid (ssDNA) can be combined together to form a SERS probe. Upon being exposed to a sample containing Staphylococcus aureus, the target aptamer in bio-functionalized Au—Ag nanoparticle-modified nanospheres will bind tightly to the S. aureus to form a stable complex, and the cDNA is released. Then the RCA template binds to the cDNA to initiate the RCA, so as to generate a large number of long single-stranded DNA repeat sequences with a specific repeat. The long single-stranded DNA repeat sequences not only significantly increase the copy number of the target DNA, but also provide abundant recognition sites for subsequent SERS detection. Subsequently, the SERS probe undergoes rapid and efficient molecular hybridization with the long single-stranded DNA repeat sequences based on complementary base pairing to further enhance the SERS signal.

Some embodiments of the present disclosure relate to a SERS-based detection method for S. aureus using a SERS-based test kit as described herein, which is performed as follows.

Step (1) Magnetic nanospheres are positively charged.

Step (2) The positively-charged magnetic nanospheres are dispersed in ultrapure water, to which the Au—Ag NPs are added and ultrasonically mixed and reacted for 2.5-3.5 h. Magnetic separation is carried out to obtain Au—Ag NP-modified nanospheres.

Step (3) The Au—Ag NP-modified nanospheres are ultrasonically dispersed in a phosphate buffered saline, to which the cDNA is added. The reaction mixture is incubated at 25-40° C. for 6-10 h, and subjected to magnetic separation. The separated product is ultrasonically dispersed in a phosphate buffered saline, to which the target aptamer is added. The reaction mixture is incubated at 25-40° C. for 30-60 min, and subjected to magnetic separation to obtain bio-functionalized Au—Ag NP-modified nanospheres.

Step (4) The Au NPs are reacted with the SERS signaling molecules under stirring for 20-40 min, and centrifuged to obtain signaling molecule-modified Au nanoparticles (Au-SM).

Step (5) The signaling molecule-modified Au nanoparticles are reacted in a 3-aminopropyltriethoxysilane aqueous solution under stirring for 10-30 min, and then the reaction mixture is added with a sodium silicate solution, reacted under stirring at 85-95° C. for 1.5-2.5 h, and subjected to standing away from light for 10-12 h, and centrifugation to obtain the silica-coated Au-SM (Au-SM/SiO₂).

Step (6) The Au-SM/SiO₂ is mixed ultrasonically with a 3-aminopropyltriethoxysilane aqueous solution for 30-40 min to obtain the amino-modified Au-SM/SiO₂, which is then reacted with a cyanuric chloride solution under stirring at room temperature for 2-3 h. The reaction mixture is added with the ssDNA and incubated at 25-40° C. for 6-10 h to obtain the ssDNA-linked Au-SM/SiO₂.

Step (7) The bio-functionalized Au—Ag NP-modified nanospheres are incubated with a to-be-detected sample containing S. aureus at 20-40° C. for 0.5-2 h. The reaction system is subjected to magnetic separation, added with the RCA template, T4 DNA ligase and T4 DNA ligase reaction buffer, adjusted to pH 7-8, and reacted at 20-40° C. for 1-3 h. Then the reaction system is subjected to magnetic separation, added with dNTPs, Phi29 DNA polymerase and Phi 29 DNA polymerase reaction buffer, incubated at 20-40° C. for 0.5-2 h, added with the ssDNA-linked Au-SMISiO₂, and incubated at 20-40° C. for 0.5-2 h. The reaction system is subjected to magnetic separation, and the separated product is detected by Raman spectrometry.

In some embodiments, the Au—Ag NPs can be prepared by conventional methods in the art. For example, the Au—Ag NPs can be prepared through the following steps: heating a chloroauric acid hydrate solution to boil; adding a sodium citrate solution followed by mixing and reaction under heating for 10-20 min; continuing to add the sodium citrate solution, and dropwise adding a silver nitrate solution; reacting the reaction system under heating for 15-30 min; after the reaction is completed, naturally cooling the reaction mixture to room temperature to obtain the Au—Ag NPs.

In some embodiments, a concentration of the chloroauric acid hydrate solution is 0.5-1.5 mM, and a volume of the chloroauric acid hydrate solution is 20-40 mL.

In some embodiments, the sodium citrate solution has a weight percentage concentration of 0.5-1.5 wt. %, and a volume of the sodium citrate solution in the first addition is 1-3 mL, and a volume of the sodium citrate solution in the second addition is 0.5-1.5 mL.

In some embodiments, a concentration of the silver nitrate solution is 0.5-2 mM, and a volume of the silver nitrate solution is 3-6 mL.

In some embodiments, the magnetic nanospheres are magnetic iron nanospheres, which can be obtained by conventional methods in the art. For example, the magnetic nanospheres can be prepared through the following steps: dissolving ferric chloride hexahydrate in ethylene glycol; adding sodium acetate and trisodium citrate followed by mixing and heating to 50-70° C.; reacting the reaction mixture under stirring for 25-35 min; after the reaction is completed, transferring the reaction mixture to a high-pressure reactor for a high-temperature reaction; and washing the reaction mixture with ethanol and ultrapure water followed by magnetic separation with a magnet to collect the magnetic iron nanospheres.

In some embodiments, a ratio of a weight of the ferric chloride hexahydrate to a weight of the sodium acetate to a weight of the trisodium citrate to a volume of the ethylene glycol is 0.9 (g): 3.7 (g): 1.5-2.0 (g): 30-50 (mL).

In some embodiments, the high-temperature reaction is carried out at 190-210° C. for 14-18 h.

In some embodiments, a volume ratio of the ethanol to the ultrapure water is 1:(0.5-1).

In some embodiments, the positively-charged magnetic iron nanospheres are obtained through the following steps: dispersing the magnetic iron nanospheres in polyethylenimine (PEI) followed by reaction under ultrasonic stirring for 1.5-2.5 h; and washing the reaction system with ethanol and ultrapure water followed by magnetic separation with a magnet to collect the positively-charged magnetic iron nanospheres (Fe MMs@PEI).

In some embodiments, the amount of the magnetic iron nanospheres is 50-100 mg.

In some embodiments, a concentration of the PEI solution is 2 mg/mL-5 mg/mL, and a volume of the PEI solution is 50-100 mL.

In some embodiments, a volume ratio of the ethanol to the ultrapure water is 1:(0.5-1).

In some embodiments, the Au—Ag nanoparticle-modified nanospheres are prepared through the following steps: dispersing the prepared positively-charged magnetic iron nanospheres (Fe MMs@PEI) in ultrapure water; adding the prepared Au—Ag NPs dispersion followed by ultrasonic mixing and reaction for 2.5-3.5 h; and subjecting the reaction mixture to magnetic separation with a magnet and washing with ethanol and ultrapure water to produce magnetic iron nanospheres decorated with Au—Ag NPs (FAA).

In some embodiments, a concentration of the Fe MMs@PEI aqueous solution is 2-5 mg/mL.

In some embodiments, a volume ratio of the Fe MMs@PEI aqueous solution to the Au—Ag NPs dispersion is 1:(2-4).

In some embodiments, a volume ratio of the ethanol to the ultrapure water is 1:(0.5-1).

In some embodiments, the bio-functionalized FAA is obtained through the following steps: ultrasonically dispersing the prepared FAA in a phosphate buffered saline (PBS) followed by adding of the cDNA probe solution for a first incubation; subjecting the incubation system to magnetic separation to obtain the cDNA-modified FAA; dispersing the cDNA-modified FAA into a phosphate buffered saline followed by adding of the target aptamer solution for a second incubation; and subjecting the incubation system to magnetic separation and washing with PBS to collect the bio-functionalized FAA.

In some embodiments, a concentration of the FAA solution is 1-2 mg/mL, and a volume of the FAA solution is 300-600 μL.

In some embodiments, a concentration of the cDNA probe solution is 10-30 M, and a volume of the cDNA probe solution is 200-400 μL.

In some embodiments, the first incubation is performed at 25-40° C. for 6-10 h.

In some embodiments, a concentration of the aptamer solution is 10 μM-30 μM, and a volume of the aptamer solution is 300-600 μL.

In some embodiments, the second incubation is performed at 25-40° C. for 30-60 min.

In some embodiments, a pH of the phosphate buffered saline is 7.2-7.4, and the amount of the phosphate buffered saline is 0.5-2 mL.

In some embodiments, the Au-SM is obtained through the following steps: heating the chloroauric acid hydrate solution to boil; adding the sodium citrate solution, followed by mixing and reaction under heating for 10-20 min; then naturally cooling the reaction mixture to room temperature to obtain the Au NP; adding a SERS signaling molecule solution to the reaction mixture followed by reaction under vigorous stirring for 20-40 min; after the reaction is completed, subjecting the reaction mixture to centrifugal separation and re-dispersion in the same volume of ultrapure water to obtain the Au-SM.

In some embodiments, a concentration of the chloroauric acid hydrate solution is 0.5-1.5 mM, and a volume of the chloroauric acid hydrate solution is 20-40 mL.

In some embodiments, the sodium citrate solution has a weight percentage concentration of 0.5-1.5 wt. %, and a volume of the sodium citrate solution is 1-3 mL.

In some embodiments, a concentration of the SERS signal molecule solution is 10⁻³-10⁻⁶ M, and a volume of the SERS signal molecule solution is 400-600 μL.

In some embodiments, the centrifugal separation is performed at 8,000-12,000 rpm for 10-15 min.

In some embodiments, the Au-SM/SiO₂ is obtained through the following steps: adding the prepared Au-SM to a 3-aminopropyltriethoxysilane aqueous solution followed by vigorous stirring for 10-30 min; dropwise adding a sodium silicate solution followed by reaction under slow stirring at 85-95° C. for 1.5-2.5 h; and subjecting the reaction mixture to standing overnight in a dark environment created by tin foil, followed by addition of ultrapure water, centrifugal separation and redispersing in an equal volume of ultrapure water to obtain the Au-SM/SiO₂.

In some embodiments, a concentration of the 3-aminopropyltriethoxysilane aqueous solution is 0.5-1.5 mM, and a volume of the 3-aminopropyltriethoxysilane aqueous solution is 0.1-0.4 mL.

In some embodiments, the sodium silicate solution has a weight percentage concentration of 0.4-0.6 wt. % with a pH of 8-9, and a volume of the sodium silicate solution is 1-4 mL.

In some embodiments, the standing is performed in the dark environment for 10-12 h.

In some embodiments, the centrifugal separation is performed at 8,000-12,000 rpm for 10-15 min.

In some embodiments, the ssDNA-linked Au-SM/SiO₂ (ssDNA-functionalized Au-SM/SiO₂) is obtained through the following steps: ultrasonically mixing the prepared Au-SM/SiO₂ with a 3-aminopropyltriethoxysilane aqueous solution for 30-40 min; after the reaction is completed, centrifugal cleaning the reaction mixture with ultrapure water followed by dispersing in an equal volume of ultrapure water to obtain an amino-modified Au-SM/SiO₂; adding a cyanuric chloride solution followed by reaction under stirring at room temperature for 2-3 h; after the reaction is completed, centrifugal cleaning the reaction mixture with phosphate buffered saline followed by dispersing into 5 mL of buffer; introducing a ssDNA solution for incubation followed by centrifugal separation, washing with phosphate buffered saline again and dispersing into an equal volume of buffer to produce ssDNA-functionalized Au-SM/SiO₂.

In some embodiments, a concentration of the 3-aminopropyltriethoxysilane aqueous solution is 0.1-0.4 mM, and a volume of the 3-aminopropyltriethoxysilane aqueous solution is 0.5-1.5 mL.

In some embodiments, a concentration of the cyanuric chloride solution is 0.5 mM-1.5 mM, and a volume of the cyanuric chloride solution is 0.5-2 mL.

In some embodiments, the incubation is performed at 25-40° C. for 6-10 h.

In some embodiments, a pH of the phosphate buffered saline is 7.2-7.4, and the amount of the phosphate buffered saline is 4-6 mL.

In some embodiments, the centrifugal separation is performed at 8,000-12,000 rpm for 10-15 min.

In some embodiments, a series of S. aureus standard solutions varying in concentration are prepared, and respectively added with the prepared bio-functionalized FAA, mixed uniformly and then incubated in a thermostatic oscillator to achieve the specific recognition of the target aptamer to S. aureus, where the cDNA is released as the initiator of RCA. After the incubation, FAA-cDNA complementary to the RCA template is obtained by magnetic separation, dispersed in a phosphate buffered saline, added with the RCA template, T4 DNA ligase and T4 DNA ligase Buffer are added, and subjected to standing for reaction. After the reaction is completed, the reaction mixture is subjected to magnetic separation to remove the unreacted RCA templates, and redispersed in a phosphate buffered saline, added with dNTPs, Phi29 DNA polymerase and Phi 29 DNA polymerase Buffer and transferred to a shaker for incubation. Then the incubation system is added with the prepared ssDNA-functionalized Au-SM/SiO₂, incubated to accomplish the complementary binding of ssDNA to the long single-stranded DNA repeat sequences obtained by RCA, and subjected to magnetic separation. The SERS spectrum of the magnetic separation product is collected by a Raman spectrometer, and linear fitting is performed between the logarithms of the concentrations of the S. aureus standard solutions and corresponding SERS signals to establish a standard curve for the detection of S. aureus content.

In some embodiments, a concentration of the RCA template is 5-15 μM, and a volume of the RCA template 5-20 μL.

In some embodiments, a concentration of the T4 DNA ligase is 80-120 U/μL, and a volume of the T4 DNA ligase is 0.5-2 μL.

In some embodiments, a volume of the T4 DNA Ligase Buffer is 10-40 μL.

In some embodiments, a concentration of the dNTPs is 5-20 nM, and a volume of the dNTPs is 2-10 μL.

In some embodiments, a concentration of the Phi29 DNA polymerase is 5-20 U/μL, and a volume of the Phi29 DNA polymerase is 1-2 μL.

In some embodiments, a volume of the Phi 29 DNA polymerase Buffer is 2-10 μL.

In some embodiments, the standing is carried out at 20-40° C. for 1-3 h.

In some embodiments, the incubation is performed at 20-40° C. for 0.5-2 h.

The disclosure will be further described below with reference to specific embodiments.

In the following embodiments, a nucleotide sequence of the RCA template consists of SEQ ID NO: 1 with 5′ end modified with a phosphate group and 3′ end modified with a hydroxyl group; a nucleotide sequence of the target aptamer consists of SEQ ID NO: 2; a nucleotide sequence of the cDNA consists of SEQ ID NO: 3 with 5′ end modified with a sulfhydryl group; and a nucleotide sequence of the ssDNA consists of SEQ ID NO: 4 with 5′ end modified with an amino group (as shown in Table 1). These sequences can be artificially synthesized by using conventional technical means in the art.

TABLE 1
Nucleotide sequence information

| | SEQ | |
|---|---|---|
| | ID | Nucleotide sequence |
| Item | NO | (5′-3′) |
| | | GCAATGGTACGGTACTTCC |
| | | TCGGCACGTTCTCAGTAGC |
| | | GCTCG |
| Target | 2 | CTGGTCATCCCACAGCTAC |
| aptamer | | GTCAAAAGTGCACGCTACT |
| | | TTGCTAA |
| cDNA | 3 | SH-TTAGCAAAGTAGCGTG |
| | | CACTPO₄- |
| Rolling | 1 | ACTTTGCTAATGGGTCAGG |
| circle | | TGCAGTGCACGCT-OH |
| amplification | | |
| template | | |
| ssDNA | 4 | NH₂-TGGGTCAGGTGC |

The reagents involved in the following embodiments are all commercially available.

HAuCl₄·3H₂O, AgNO₃, sodium citrate (Na₃CIT), 4-nitrothiophenol (4-NTP), polyethylene glycol (Mw=6000), sodium acetate (NaAc, purity ≥99.0%), polyethyleneimine (PEI), 2,4,6-trichloro-1,3,5-triazine (TCT), Na₂SiO₃, 3-(aminopropyl) triethoxysilane (APTES), Acetonitrile, ethylene glycol (C₂H₆O₂, purity ≥99.5%), Ethanol (C₂H₆O), FeCl₃·6H₂O (purity ≥99.0%), rhodamine 6G (R6G), PBS (pH=7.4), Luria Brtani (LB) Culture medium, Agarose, and 50×Tris-Acetate-EDTA (TAE) buffer are purchased from Sinopharm Chemical Reagent Co., Ltd (Shanghai, China). dNTPs, T4 DNA ligase, and phi29 DNA polymerase are purchased from Thermo Fisher Scientific Inc. Pathogenic strains such as S. aureus (ATCC 29213), Listeria monocytogenes (ATCC 19115), Salmonella typhimurium (ATCC 50761), Bacillus subtilis (ATCC 6633) and Escherichia coli (ATCC 25922) are purchased from the American Type Culture Collection (ATCC). Pseudomonas aeruginosa (BNCC 125486) is purchased from BeNa Culture Collection (Suzhou, Jiangsu, China). Vibrio parahaemolyticus (CICC 10552) is purchased from China Center of Industrial Culture Collection. All chemicals and reagents are of analytical grade, and do not require further purification. All experiments are performed in ultrapure water.

### Example 1

The present example provided a SERS-based detection method of S. aureus, including the following steps.

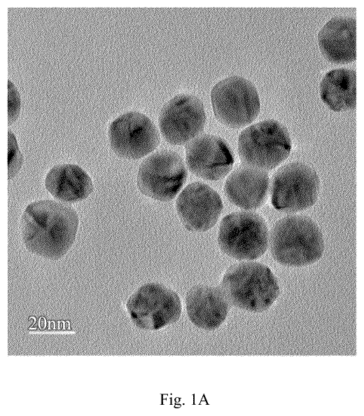

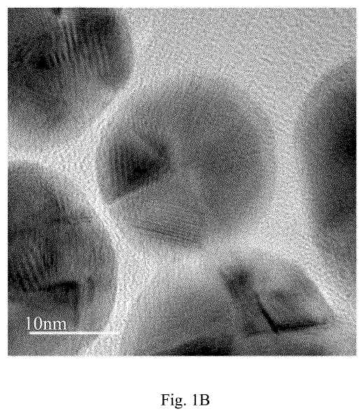

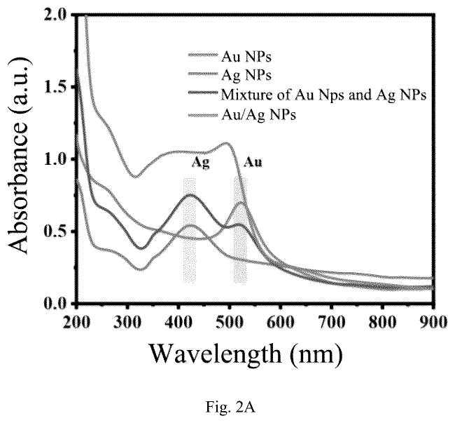

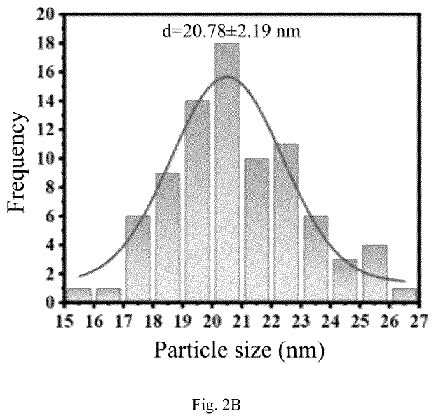

Step (1) 25 mL of a chloroauric acid hydrate solution (1 mM) was heated to boil, added with 2.5 mL of a 1 wt. % sodium citrate solution, mixed, reacted under heating for 15 min, and further added with 1 mL of the 1 wt. % sodium citrate solution. The reaction mixture was dropwise added with 4 mL of a silver nitrate solution (1.07 mM), reacted under heating for 20 min and naturally cooled to room temperature to obtain the Au—Ag NPs, which were characterized by the transmission electron microscope (TEM) images in FIGS. 1A-B. The nanoparticles showed a clear core-shell structure with a silver core and a thin gold shell wrapped around the silver core, and had uniform size and regular shape. FIGS. 2A-2B respectively showed ultraviolet (UV) spectral data and particle size data of the prepared Au—Ag NPs. The UV analysis showed that the Au—Ag NPs had specific absorption peaks at 388 and 498 nm, both of which showed a blue shift compared to the absorption peaks of Au NPs (420 nm) and Ag nanoparticles (Ag NPs) (520 nm). In addition, the particle size measurement results revealed that the particle size and distribution range was 20.78±2.19 nm.

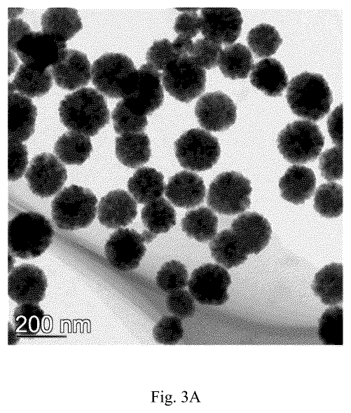

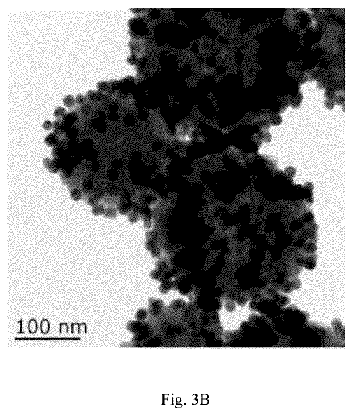

Step (2) 0.9 g of ferric chloride hexahydrate was dissolved in 40 mL of ethylene glycol, to which 3.7 g of sodium acetate and 1.7 g of trisodium citrate were added. The reaction mixture was mixed, heated to 65° C. and reacted under stirring for 30 min. After the reaction was completed, the reaction mixture was transferred to a high-pressure reactor, and reacted at 200° C. for 16 h. After the reaction was completed, the reaction mixture was washed with ethanol and ultrapure water (a volume ratio of the ethanol to the ultrapure water was 1:0.6), and magnetically separated by a magnet to obtain iron magnetic nanospheres (Fe MMs). FIG. 3A showed the TEM image of Fe MMs, from which it can be observed that the microspheres were morphologically regular and possessed excellent structural characteristics.

Step (3) 100 mg of the Fe MMs prepared in step (2) was added in 75 mL of a polyethyleneimine solution (5 mg/mL), and reacted under ultrasonic stirring for 2 h. After the reaction was completed, the reaction product was washed with ethanol and ultrapure water (the volume ratio of ethanol to ultrapure water was 1:0.6) and subjected to magnetic separation with a magnet to obtain positively-charged Fe MMs (i.e., Fe MMs@PEI).

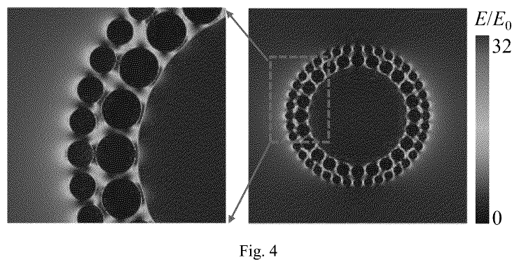

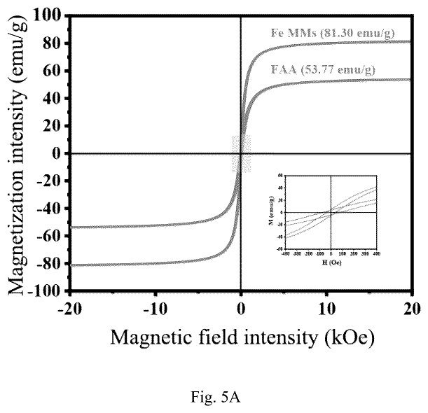

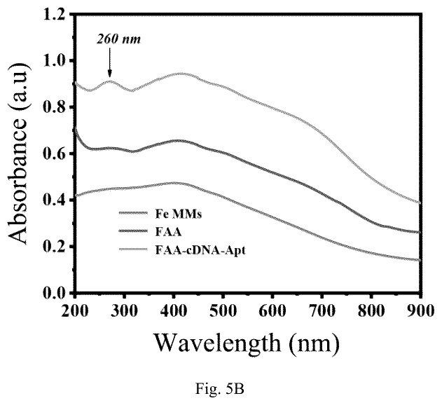

Step (4) The Fe MMs@PEI was dispersed uniformly in ultrapure water to form a dispersion with a concentration of 5 mg/mL. Then, 5 mL of the dispersion was mixed with 15 mL of the Au—Ag NPs solution prepared in step (1), and reacted under ultrasonication for 3 h. After the reaction was completed, the reaction mixture was subjected to magnetic separation with the magnet, and washed with ethanol and ultrapure water at a volume ratio of 1:0.6 to obtain the Au—Ag nanoparticle-modified iron nanospheres (FAA). FIG. 3B was a TEM image of the FAA, showing that the Au—Ag NPs tightly adhered to the surface of the magnetic nanospheres, and formed a uniform and dense coating. FIG. 4 showed the finite-difference time-domain computational data of the electric field strength distribution of FAA/Au NPs. The “hot spots” of FAA/Au NPs were mainly distributed in the nanogaps, which indicated that the prepared SERS nanotags could generate strong electric fields in the gap between FAA and Au NPs to offer excellent SERS activity. FIG. 5A showed the magnetic hysteresis loop characterization of Fe MMs and FAA, which demonstrated the effect of nanoparticle modification on the magnetic properties of magnetic nanospheres, and clearly illustrated the comparison between magnetic hysteresis loops of the Fe MMs and FAA in the electric field range of −20 kOe to +20 kOe under room temperature conditions. Pure iron magnetic nanospheres exhibited excellent magnetic properties with the magnetization saturation (Ms) reaching 81.30 emu/g and coercivity (Hc) reaching 19 Oe. However, after modified with the Au—Ag NPs, the Ms value was reduced to 53.77 emu/g. Notably, the introduction of Au—Ag NPs increased the coercivity to 35 Oe, which could be attributed to the fact that the introduction of Au—Ag NPs increased the magnetic inhomogeneity of the microsphere surface, such that more energy was required when the microspheres withstood the change of the magnetization state caused by the external magnetic field.

Step (5) The FAA prepared in step (4) was ultrasonically dispersed in a phosphate buffered saline to obtain a dispersion with a concentration of 1 mg/mL. 400 L of the dispersion was incubated with an amino-modified cDNA solution (350 μL, 15 μM) at 35° C. for 8 h. After the incubation was completed, the incubation system was subjected to magnetic separation to obtain the cDNA-decorated FAA, which was dispersed in 400 μL of a phosphate buffered saline (pH=7.4), added with 400 μL of a target aptamer solution (15 μM) and incubated at 35° C. for 40 min. After the incubation was completed, the incubation system was subjected to magnetic separation, and the separated product was washed with a phosphate buffered saline, and dispersed in 400 L of phosphate buffer to obtain the bio-functionalized FAA. FIG. 5B showed the UV adsorption of Fe MMs, FAA and bio-functionalized FAA (FAA-cDNA-Apt). The UV spectrum of bio-functionalized FAA showed a new adsorption peak at 260 nm, whereas no obvious peak was observed in spectra of Fe MMs and FAA, suggesting that the aptamer and its complementary chain were successfully assembled on the FAA surface.

Step (6) 25 mL of a chloroauric acid hydrate solution (1 mM) was heated to boil, to which 2.5 mL of a 1 wt. % sodium citrate solution was added. The reaction mixture was mixed, reacted under heating for 15 min, and naturally cooled to room temperature to obtain Au NPs. The reaction mixture was added with 500 μL of a signaling molecule solution (10⁻⁴ M), reacted under vigorous stirring for 30 min, centrifuged at 10,000 rpm for 10 min and dispersed in 25 mL of ultrapure water to obtain signaling molecule-modified Au nanoparticles (Au-SM).

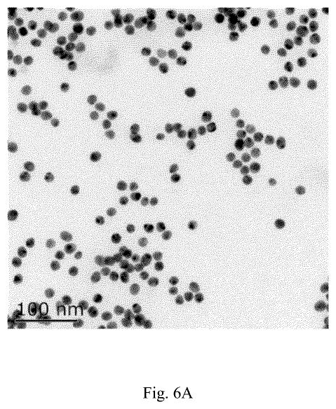

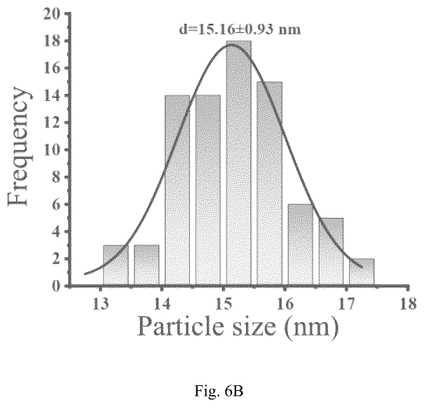

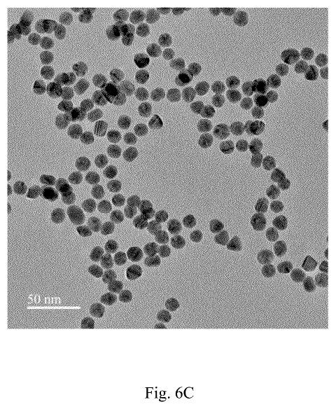

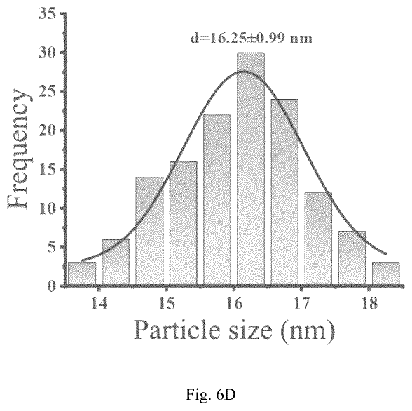

Step (7) 25 mL of the Au-SM prepared in step (6) was added with 0.2 mL of a 3-aminopropyltriethoxysilane aqueous solution (1 mM) and stirred vigorously for 15 min. Then the reaction mixture was dropwise added with 2 mL of a 0.54 wt. % sodium silicate solution (pH=8), and reacted at 90° C. under gentle stirring for 2 h. The reaction system was allowed to stand for 10 h in a dark environment created by tin foil, added with ultrapure water, centrifuged at 10,000 rpm for 10 min, and dispersed in 25 mL of ultrapure water to obtain SiO₂-coated Au-SM (Au-SM/SiO₂). FIGS. 6A-6D showed the TEM characterization data and particle size distribution of the prepared Au NPs and Au-SM/SiO₂. The Au NPs and Au-SM/SiO₂ showed a clear spherical or near-spherical shape with clear particle edges and uniform contrast. The particle size measurement was performed by image analysis software, and the results revealed that the particle sizes of Au NPs and Au-SM/SiO₂ were mainly within 15.16-0.93 nm and 16.25-0.99 nm, respectively.

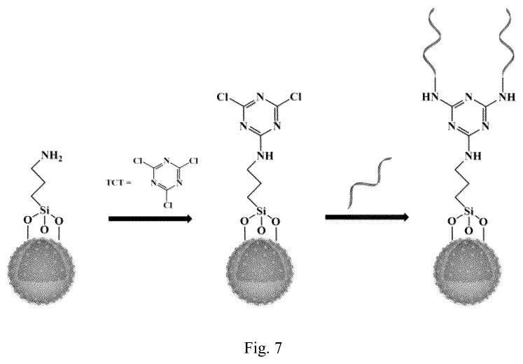

Step (8) 25 mL of the Au-SM/SiO₂ prepared in step (7) was dispersed in a 3-aminopropyltriethoxysilane aqueous solution (0.1 mM) and ultrasonically mixed for 30 min. After the reaction was completed, the reaction mixture was subjected to centrifugal washing with ultrapure water and dispersed in 25 mL of ultrapure water to obtain amino-modified Au-SM/SiO₂. Then the amino-modified Au-SM/SiO₂ was added with 1 mL of a cyanuric chloride solution (1 mM) and reacted under stirring at room temperature for 2 h. After the reaction was completed, the reaction mixture was subjected to centrifugal washing with a phosphate buffered saline and dispersed into 5 mL of PBS, to which a ssDNA solution (500 μL, 15 μM) was introduced. The reaction mixture was incubated, centrifuged at 10,000 rpm for 10 min, washed with PBS and dispersed into 5 mL of PBS to prepare ssDNA-functionalized Au-SM/SiO₂. FIG. 7 showed a synthetic route of the ssDNA-functionalized Au-SM/SiO₂.

Step (9) A series of S. aureus standard solutions varying in concentration (within a range of 3.6-36×10⁷ cfu/mL) were prepared, and 50 μL of individual standard solutions were respectively added with 50 μL of the bio-functionalized FAA prepared in step (5), diluted to 200 μL with PBS, mixed uniformly and incubated in a thermostatic shaker at 35° C. for 1 h to achieve the specific recognition between the target and the aptamer strand, where the cDNA was released and played an initiator role for the RCA. After the incubation was completed, the incubation system was subjected to magnetic separation to obtain FAA-cDNA complementary to the RCA template, which was dispersed in 200 μL of PBS, added with the RCA template (10 μL, 10 PM), T4 DNA ligase (1 μL, 100 U/μL) and T4 DNA ligase Buffer (20 μL) were added, and subjected to standing at 35° C. for 2 h for reaction. After the reaction was completed, the unreacted RCA template was removed by magnetic separation, and the separation product was dispersed in 200 μL of PBS, added with 5 μL of dNTPs, 1 μL of Phi29 DNA polymerase (10 U/μL) and 5 μL of Phi29 DNA polymerase Buffer and incubated in a shaker at 35° C. for 1 h. Then, the incubation system was added with the ssDNA-functionalized Au-SM/SiO₂ (10 μL) obtained from step (S8) and incubated at 35° C. for 1 h to complete the complementary binding between ssDNA and the long single-stranded DNA repeat sequence generated by RCA. The incubation system was subjected to magnetic separation. The SERS spectrum of the magnetic separation product was collected by Raman spectroscopy, and linearly fitting was performed between logarithms of the concentrations of the S. aureus standard solutions and the corresponding SERS signals.

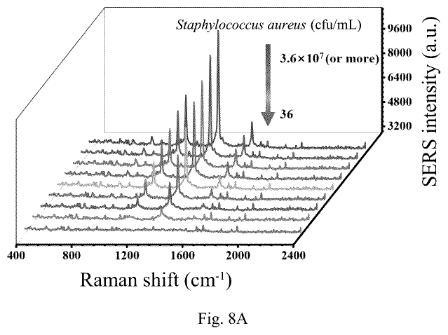

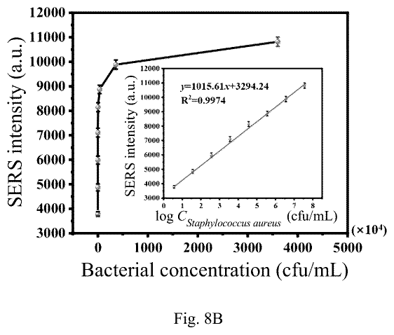

The signaling molecule used herein was 4-nitrothiophenol, and its SERS signal intensity at 1334.1 cm⁻¹ was attributed to the C—N stretching and the symmetric deformation of CH₃, and gradually increased with the concentration of S. aureus. The linear relationship between the SERS signal intensity and the S. aureus concentration was represented by the standard curve y=1015.61x−3294.24, R²=0.9974. In addition, the limit of detection (LOD) was calculated as 2.0 cfu/mL (LOD=3δ/S, where δ was the relative standard deviation of the evaluated values from 10 blank samples, and S was the slope of the calibration curve). The experiment results were displayed in FIGS. 8A-8B, where FIG. 8A showed typical SERS spectra of various concentrations of S. aureus recorded by the signal amplification platform; and FIG. 8B demonstrated the functional relationship between the concentration of S. aureus and the SERS intensity, and provided a calibration curve for the detection of S. aureus.

### Example 2

### Optimization of the Reaction Conditions

1. Modification of Iron Magnetic Nanospheres (Fe MMs) with Au—Ag NPs

The Au—Ag NPs on the surface of Fe MMs were optimized to promote the effective ligation of cDNA sequences to ensure the directional and stable arrangement of cDNA sequences on the FAA surface, which was conducive to the subsequent complementary pairing of aptamer strands with DNA sequences. In addition, the optimization of Au—Ag NPs can affect indirectly the distribution and accessibility of the aptamer strands on the FAA surface, enabling the aptamer strand to bind to S. aureus more efficiently.

Fe MMs were first amino-functionalized as follows. Specifically, 0.04 g of Fe MMs was dispersed in 20 mL of an aqueous solution containing 0.1 g of PEI, ultrasonicated for 2 h and washed three times with ethanol and ultrapure water, respectively. The obtained Fe MMs@PEI material was dispersed in 40 mL of ultrapure water (1 mg/mL), and the Au—Ag NPs were assembled onto the Fe MMs@PEI via electrostatic adsorption. In order to arrive at the FAA with the optimal SERS-activity, the amino-modified Fe MMs were respectively with different volumes of the Au—Ag NPs (10, 20, 30, and 40 mL), and continuously stirred for 3 h, and subjected to magnetic separation in the presence of an external magnetic field to collect the FAA material, which was washed in turn with ethanol and ultrapure water, and redispersed in 40 mL of PBS (pH=7.4).

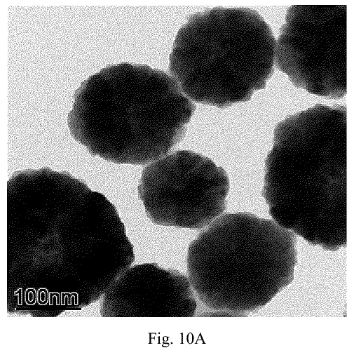

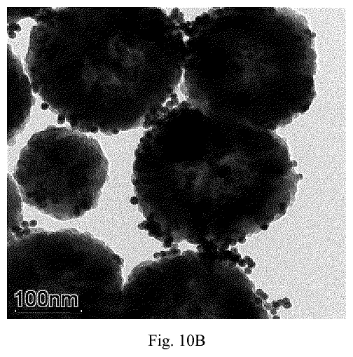

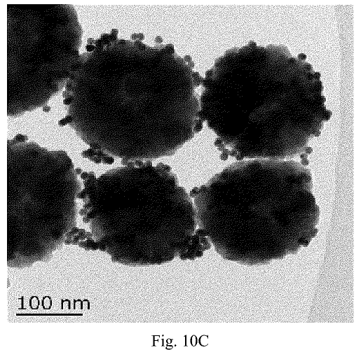

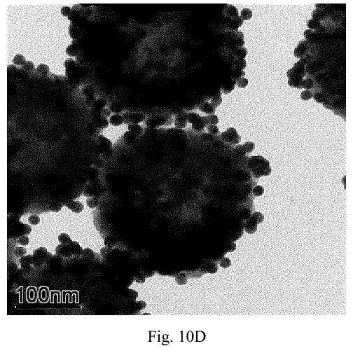

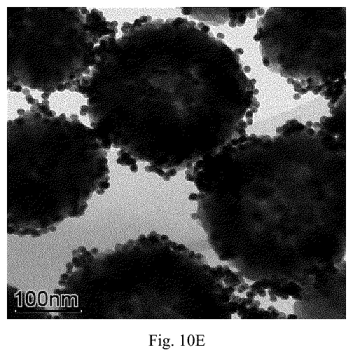

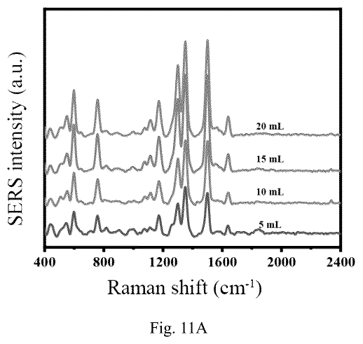

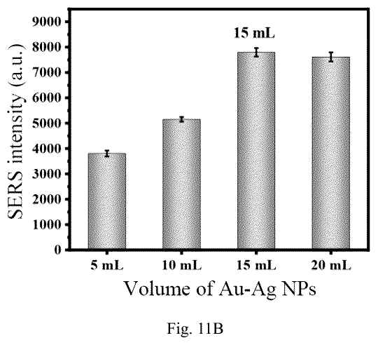

FIGS. 10A-10E respectively showed TEM images of FAA materials obtained under different volumes of Au—Ag NPs. The series of FAA materials were further analyzed by SERS (according to the method described in Example 1), and the SERS spectra and intensity were respectively shown in FIGS. 11A-11B.

In combination with the calculation results of enhancement factor (EF), it was further found that the FAA material synthesized in the presence of 30 mL of Au—Ag NPs exhibited the highest EF value (2.65×10⁷), as shown in Table 2.

TABLE 2
EF of Fe MMs modified with different volumes of Au—Ag NPs

| Au—Ag NPs | I_{RS} | I_{SERS} | C_{RS} | C_{SERS} | |
|---|---|---|---|---|---|
| volume (mL) | (a.u.) | (a.u.) | (mol/L) | (mol/L) | EF |
| 5 | 68.54 | 121.76 | 0.14 | 1.6 × 10⁻² | 0.16 × 10² |
| 10 | 68.54 | 349.28 | 0.14 | 1.6 × 10⁻⁴ | 5.10 × 10⁴ |
| 15 | 68.54 | 745.19 | 0.14 | 1.6 × 10⁻⁶ | 9.63 × 10⁵ |
| 20 | 68.54 | 726.03 | 0.14 | 1.6 × 10⁻⁶ | 9.27 × 10⁵ |

Note:
C_{RS} and I_{RS} were the concentration of Rhodamine 6G (R6G) and the spectral intensity value at 1509 cm⁻¹, respectively, in the absence of FAA. C_{SERS} and I_{SERS} were the concentration of R6G and the spectral intensity value at 1509 cm⁻¹, respectively, in the presence of FAA.

Therefore, regarding the surface modification of Fe MMs, the optimal volume of Au—Ag NPs was determined to be 30 mL.

### 2. Investigation on pH of the RCA System, RCA Temperature, T4 DNA Ligase, Phi29 DNA Polymerase, and Reaction Time

### (1) Optimization of pH and Temperature for RCA

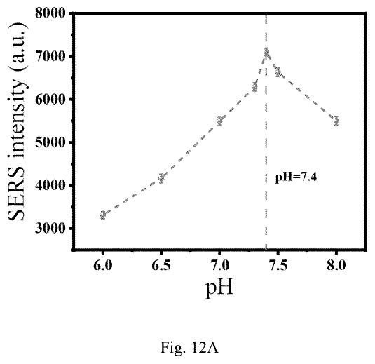

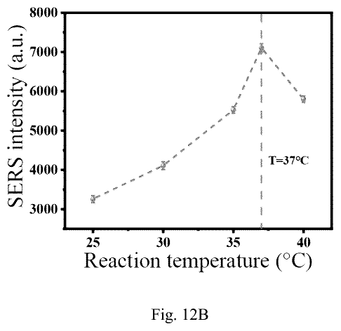

Influences of RCA pH and temperature on the SERS intensity were investigated herein in the presence of 4.2×10³ cfu/mL of S. aureus, and other experimental conditions were identical to those in Example 1. As shown in FIG. 12A, the SERS intensity was minimum at pH 6, increased at pH 7 compared to pH 6.5, and then reached the maximum at pH 7.4. The SERS intensity started to decline when the pH continuously rose from 7.4. Therefore, pH 7.4 was chosen as the optimal condition. FIG. 12B showed the change of SERS intensity versus reaction temperature of the RCA, from which it can be seen that the maximum signal intensity appeared at 37° C., and thus 37° C. was chosen as the experimental temperature.

### (2) Amount of Phi29 DNA Polymerase and T4 DNA Ligase

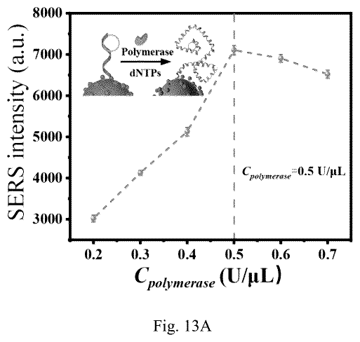

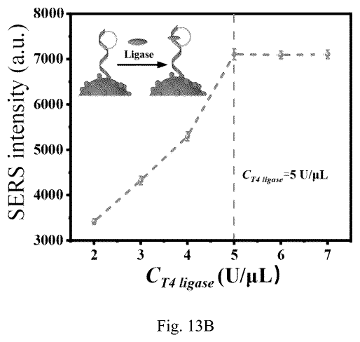

The amounts of phi29 DNA polymerase and T4 DNA ligase were optimized in the presence of 4.2×10³ cfu/mL of S. aureus, and other experimental conditions were identical to those in Example 1. As shown in FIG. 13A, the SERS intensity increased rapidly as the amount of phi29 DNA polymerase was increased from 0.2 U/μL to 0.5 U/μL, and when the concentration exceeded 0.5 U/μL, the SERS intensity showed a slight decline. Therefore, 0.5 U/μL was selected as the optimal concentration of phi 29 DNA polymerase. Similarly, FIG. 13B showed the effect of T4 DNA ligase concentration on the SERS intensity. As can be seen from the FIG. 13B, 5 U/μL is considered as the optimal concentration of T4 ligase.

### (3) Optimization of RCA Reaction Time and Probe Hybridization Time

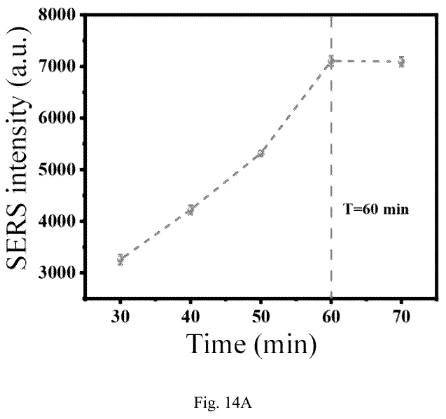

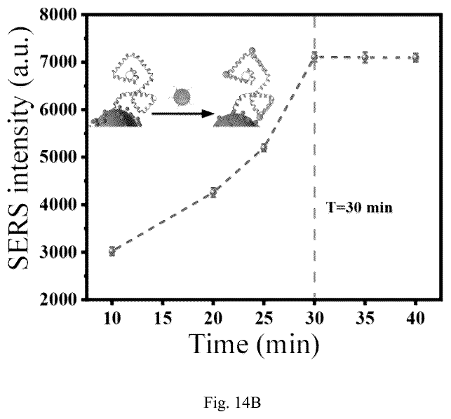

As shown in FIG. 14A, the SERS intensity increased rapidly as the RCA reaction time was extended from 30 min to 1 h in the presence of optimal concentrations of phi29 polymerase and substrate. When the reaction time exceeded 1 h, the SERS intensity slightly decreased and tended to be stable. Therefore, 1 h was chosen as the optimal reaction time for the RCA in the subsequent experiments. The hybridization time of the RCA products with the ssDNA-functionalized Au-SM/SiO₂ signal probe was further assessed. As shown in FIG. 14B, an enhancement of the RCA performance was observed at the optimal time of 30 min.

### Example 3

### Specificity Assessment

In order to comprehensively assess the selectivity of the method developed herein for the detection of S. aureus, different species of pathogenic bacteria (E. coli, V parahaemolyticus, B. subtilis, S. salmonicida, L. monocytogenes, and P. aeruginosa) were introduced into the detection system to simulate the complex environment in the real biological samples. The concentration of S. aureus used herein was 3.6×10³ cfu/mL, while other pathogenic bacteria were present at an excess level of 10⁶ cfu/mL.

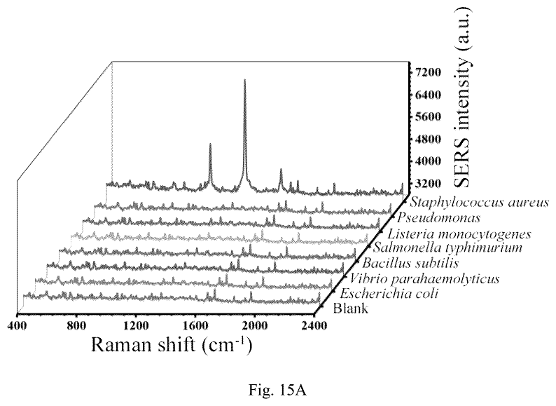

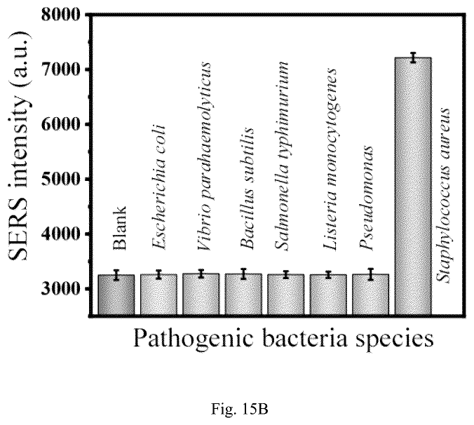

The samples were tested according to the method described in Example 1, and the results were shown in FIGS. 15A-15B. The excessive presence of other common pathogens had no significant effect on the selectivity of the proposed SERS-based method to S. aureus, and the SERS signal intensity of S. aureus was much higher than that of the other six interfering pathogens, which sufficiently confirmed the excellent applicability and specificity of the proposed method for the detection of S. aureus.

### Example 4

### Practical Application in Food Samples

A practical sample (e.g., water, milk, and fish) was added with a suspension with known concentration of S. aureus. The spiked recovery results obtained by respectively spiking different concentrations of S. aureus standard solutions (1.0×10², 1.0×10³ and 1.0×10⁴ cfu/mL) into the test sample were presented in Table 3. The results showed excellent consistency between the spiked concentration and the detected content in all of the water, milk and fish samples, with recoveries ranging from 92.44% to 107.82%, as shown in Table 3.

TABLE 3
Analytical application and validation of the SERS-based detection method for S. aureus

| | | Present method | | | Plate counting method | | | |
|---|---|---|---|---|---|---|---|---|
| | Spiked | Detection | | | Detection | | | |
| | concentration | value ± SD | recovery | CVs | value ± SD | recovery | CVs | |
| Samples | (cfu/mL) | (cfu/mL) | (%) | (%) | (cfu/mL) | (%) | (%) | p |
| Water | 1.0 × 10² | (0.96 ± 0.04) × 10² | 96.03 | 1.89 | (1.00 ± 0.02) × 10² | 100.00 | 2.03 | 0.68 |
| | 1.0 × 10³ | (1.02 ± 0.05) × 10³ | 102.31 | 2.01 | (0.98 ± 0.03) × 10³ | 97.84 | 4.18 | |
| | 1.0 × 10⁴ | (0.94 ± 0.02) × 10⁴ | 94.02 | 1.73 | (1.02 ± 0.05) × 10⁴ | 102.31 | 6.42 | |
| Milk | 1.0 × 10² | (1.01 ± 0.04) × 10² | 101.26 | 6.09 | (0.95 ± 0.04) × 10² | 95.00 | 3.12 | 0.59 |
| | 1.0 × 10³ | (0.98 ± 0.03) × 10³ | 97.86 | 1.46 | (0.97 ± 0.03) × 10³ | 97.10 | 2.80 | |
| | 1.0 × 10⁴ | (0.99 ± 0.04) × 10⁴ | 99.41 | 7.22 | (1.03 ± 0.04) × 10⁴ | 103.36 | 2.13 | |
| Fish | 1.0 × 10² | (0.96 ± 0.03) × 10² | 96.35 | 3.87 | (0.97 ± 0.03) × 10² | 97.35 | 0.49 | 0.72 |
| | 1.0 × 10³ | (1.03 ± 0.05) × 10³ | 103.12 | 1.29 | (0.94 ± 0.02) × 10³ | 94.83 | 2.11 | |
| | 1.0 × 10⁴ | (0.97 ± 0.06) × 10⁴ | 97.43 | 1.46 | (0.99 ± 0.05) × 10⁴ | 99.42 | 2.32 | |

SD: Standard Deviation; CVs: Coefficient of Variation.
p: two-tailed t-test results between the method of the present disclosure and the plate counting method.
p > 0.05: no significant difference.

Table 3 showed the statistical comparison of the results obtained by the method of the present disclosure with those obtained by the traditional plate counting method in the detection of the test sample contaminated with the same concentration of S. aureus. The t-test results showed that there were no significant differences between the two methods (p>0.05). Therefore, it can be concluded that the test method of the present disclosure has high detection accuracy and precision, and can be used for routine analysis of S. aureus in the environmental, dairy and food samples.

Various technical features of the embodiments described above can be combined arbitrarily as long as there is no contradiction, and for the purpose of brevity, all possible combinations have not been described herein.

Described above are merely several embodiments of the present disclosure, which are described in a more specific and detailed manner, but are not intended to limit the scope of the present disclosure. It should be understood that various changes or improvements made by those of ordinary skill in the art without departing from the spirit of the present disclosure shall fall within the scope of the present disclosure defined by the appended claims.

What is claimed is:

1. A surface-enhanced Raman scattering (SERS)-based test kit for Staphylococcus aureus, comprising:
a magnetic nanosphere;
a gold-silver core-shell nanoparticle (Au—Ag NP);
a gold nanoparticle (Au NP);
a SERS signaling molecule;
a rolling circle amplification (RCA) template;
a target aptamer;
a complementary deoxyribonucleic acid (cDNA); and
a single-stranded deoxyribonucleic acid (ssDNA);
wherein a nucleotide sequence of the RCA template consists of SEQ ID NO: 1 with 5′ end modified with a phosphate group and 3′ end modified with a hydroxyl group;
a nucleotide sequence of the target aptamer consists of SEQ ID NO: 2;
a nucleotide sequence of the cDNA consists of SEQ ID NO: 3 with 5′ end modified with a sulfhydryl group; and
a nucleotide sequence of the ssDNA consists of SEQ ID NO: 4 with 5′ end modified with an amino group.

2. The SERS-based test kit of claim 1, wherein the magnetic nanosphere is an iron nanosphere.

3. The SERS-based test kit of claim 1, wherein a particle size of the Au—Ag NP is 15-25 nm;
a particle size of the magnetic nanosphere is 165-175 nm; and/or
a particle size of the Au NP is 10-20 nm.

4. The SERS-based test kit of claim 1, wherein the SERS signaling molecule is selected from the group consisting of 4-nitrobenzenethiol, 4-mercaptobenzoic acid, 4-aminobenzenethiol and a combination thereof.

5. The SERS-based test kit of claim 1, wherein the SERS-based test kit further comprises at least one of T4 DNA ligase, deoxyribonucleotide triphosphates (dNTPs) and Phi29 DNA polymerase.

6. A SERS-based detection method for Staphylococcus aureus using the SERS-based test kit of claim 1, comprising:
(1) charging the magnetic nanosphere positively to obtain a positively-charged magnetic nanosphere;
(2) dispersing the positively-charged magnetic nanosphere in ultrapure water to obtain a first dispersion, and adding the Au—Ag NP to the first dispersion followed by ultrasonic reaction for 2.5-3.5 h and magnetic separation to obtain an Au—Ag nanoparticle-modified nanosphere;
(3) ultrasonically dispersing the Au—Ag nanoparticle-modified nanosphere in a first phosphate buffered saline to obtain a second dispersion; adding the cDNA to the second dispersion followed by incubation at 25-40° C. for 6-10 h and magnetic separation to collect a separated product; ultrasonically dispersing the separated product into a second phosphate buffered saline to obtain a third dispersion; and adding the target aptamer to the third dispersion followed by incubation at 25-40° C. for 30-60 min and magnetic separation to obtain a bio-functionalized Au—Ag nanoparticle-modified nanosphere;
(4) reacting the Au NP with the SERS signaling molecule under stirring for 20-40 min followed by centrifugal separation to obtain a signaling molecule-modified Au NP (Au-SM);
(5) reacting the Au-SM with a 3-aminopropyltriethoxysilane aqueous solution under stirring for 10-30 min to obtain a reaction product, and reacting the reaction product with a sodium silicate solution under stirring at 85-95° C. for 1.5-2.5 h followed by standing away from light for 10-12 h, and centrifugal separation to obtain a silica-coated Au-SM (Au-SM/SiO₂);
(6) ultrasonically mixing the Au-SM/SiO₂ with a 3-aminopropyltriethoxysilane aqueous solution for 30 min-40 min to obtain an amino-modified Au-SM/SiO₂, and reacting the amino-modified Au-SM/SiO₂ with a cyanuric chloride solution at room temperature under stirring for 2-3 h followed by addition of the ssDNA and incubation at 25-40° C. for 6-10 h to obtain a ssDNA-linked Au-SM/SiO₂; and
(7) incubating the bio-functionalized Au—Ag nanoparticle-modified nanosphere obtained in step (3) with a to-be-detected sample containing Staphylococcus aureus at 20-40° C. for 0.5-2 h followed by magnetic separation to obtain a first separated product; adding the RCA template, a T4 DNA ligase and a T4 DNA ligase reaction buffer to the first separated product to obtain a reaction system; adjusting the reaction system to pH 7-8 followed by reaction at 20-40° C. for 1-3 h and magnetic separation to obtain a second separated product; adding dNTPs, a Phi29 DNA polymerase and a Phi29 DNA polymerase reaction buffer to obtain an incubation system; incubating the incubation system at 20-40° C. for 0.5-2 h followed by addition of the ssDNA-linked Au-SM/SiO₂, incubation at 20-40° C. for 0.5-2 h and magnetic separation to obtain a third separated product; and detecting the third separated product using a Raman spectrometer.

7. The SERS-based detection method of claim 6, wherein in step (7), the reaction system is adjusted to pH 7.3-7.5.

8. The SERS-based detection method of claim 6, wherein in step (7), the reaction system is reacted at 35-40° C.

9. The SERS-based detection method of claim 6, wherein in step (7), a final concentration of the Phi29 DNA polymerase in the incubation system is 0.4-0.7 U/μL; and/or
a final concentration of the T4 DNA ligase in the reaction system is 4-7 U/μL.

10. A biosensor, comprising:
the SERS-based test kit of claim 1.


SEQUENCE LISTING (4 sequences)

SEQ ID NO: 1 - DNA, 32 bp, synthetic construct
  source 1..32: mol_type = other DNA
actttgctaatgggtcaggtgcagtgcacgct

SEQ ID NO: 2 - DNA, 88 bp, synthetic construct
  source 1..88: mol_type = other DNA
gcaatggtacggtacttcctcggcacgttctcagtagcgctcgctggtcatcccacagctacgtcaaaagtgcacgctactttgctaa

SEQ ID NO: 3 - DNA, 20 bp, synthetic construct
  source 1..20: mol_type = other DNA
ttagcaaagtagcgtgcact

SEQ ID NO: 4 - DNA, 12 bp, synthetic construct
  source 1..12: mol_type = other DNA
tgggtcaggtgc